(19) United States
(12) Patent Application Publication
(10) Pub. No.: US 2021/0016079 A1
(43) Pub. Date: Jan. 21, 2021

(54) ELECTRICAL STIMULATION SYSTEM AND METHODS FOR LIMB CONTROL

(71) Applicant: Vanderbilt University, Nashville, TN (US)

(72) Inventors: Andrew Ekelem, Nashville, TN (US); Michael Goldfarb, Nashville, TN (US)

(21) Appl. No.: 16/965,898

(22) PCT Filed: Feb. 11, 2019
(86) PCT No.: PCT/US2019/017531

§ 371 Date: Jul. 29, 2020

Related U.S. Application Data
(60) Provisional application No. 62/628,393, filed on Feb. 9, 2018.

(51) Int. Cl. A61N 1/04; A61N 1/36; A61G 5/06

(52) U.S. Cl. CPC A61N 1/0456; A61G 5/06; A61N 1/36031; A61N 1/0452

(57) ABSTRACT

The present disclosure provides various systems and methods for assisting with lower limb movement. An exemplary system can include a plurality of wearable modules and a controller. Each of the plurality of wearable modules can include a stimulator and a sensor. The controller can be configured to detect a desired activity based on data from the sensors on the wearable modules. The controller can be further configured to cause the stimulator to provide electrical stimulation to assist with the desired activity.

### CROSS REFERENCE TO RELATED APPLICATIONS

This application claims priority to and the benefit of U.S. Provisional Patent Application No. 62/628,393, filed Feb. 9, 2018, entitled “ELECTRICAL STIMULATION SYSTEM AND METHODS FOR LIMB CONTROL,” the contents of which are herein incorporated by reference in their entireties.

### FIELD OF INVENTION

The present disclosure relates to a modular lower-limb system to assist with lower limb movement.

### BACKGROUND OF INVENTION

Millions of people have neurological impairments affecting lower limb movement. These neurological impairments arise from birth defects, strokes, spinal cord injury, and other events that leave lasting damage causing paralysis or with paresis. Neurological impairments lead to restricted movement and a more sedentary life; this decrease in mobility often leads to secondary health and medical complications that can be further debilitating or even life-threatening. Moreover, walking after neurological impairment can be incredibly challenging, and conventional systems do not provide comprehensive mobility assistance. Typically, conventional systems provide isolated methods of assistance but cannot accommodate different activities. For example, a user may need a first system for gait adjustment, another system for tremor reduction, another system for cycling, and so on.

Many of these conventional systems have additional drawbacks. For example, conventional systems for gait assistance often use an orthosis to provide stability and orientation for users. An orthosis, while helpful in the short-term to increase a user's mobility, ultimately restricts range of motion and fosters the user's dependency on the device. Additionally, these devices often provide bulky assistance, hindered by a complicated system of supports, wires, and mechanisms.

Furthermore, clonus can be a symptom of neurologic impairment, and is especially common during wheelchair propulsion. Clonus is a symptom of neurologic impairment termed spasticity. The neurological basis of clonus involves a stretch reflex; this stretch can be modulated by central nervous system mechanisms that make the reflex more or less sensitive to extrinsic input. For example, when a wheelchair user propels over a small bump, the user's calf muscles may undergo a stretch and possibly initiate clonus. Once initiated, clonus can be self-excitatory and continue until physically interrupted. Conventional systems and methods for treating clonus include primarily pharmacological interventions and therapeutic stretching. If clonus persists, surgical interventions may be required. Some conventional orthosis fail to reduce clonus occurrences, with any notable success.

Systems and methods are needed which can provide more comprehensive support for a user with neurological impairments.

### SUMMARY

The various examples of the present disclosure are directed towards a system including at least one wearable module and a controller. The at least one wearable module includes at least one stimulator and at least one inertial sensor. The at least one stimulator includes a pair of electrodes positioned adjacent to human tissue of a lower limb and causes a plurality of action potentials at the human tissue. The at least one inertial sensor receives inertial data of the wearable module. The controller is communicatively coupled to the stimulator and the inertial sensor. The controller determines a desired activity and a trait of the desired activity based on the received inertial data. The controller applies at least one action potential from the plurality of action potentials to the human tissue, via the at least one stimulator, for a selected duration. The applying is based on the desired activity, the at least one trait, and the inertial data.

Another embodiment of the present disclosure provides for an apparatus to reduce pathological lower limb oscillation. The apparatus includes at least one accelerometer, a computing device, an electrical stimulation unit, and a controller. The accelerometer is positioned adjacent to a lower limb. The computing device is configured to (1) receive sensor data from the accelerometer, (2) process the sensor data to provide a computed inertial measurement, and (3) determine when the computed inertial measurement passes a first threshold level. The electrical stimulation unit is a pair of electrodes and is configured to stimulate ankle dorsiflexion of the lower limb. The controller is communicatively coupled to the commuting device and is configured to activate the electrical stimulation unit. Activating the electrical stimulation unit is based on whether the computed inertial measurement passed the first threshold inertial level.

Another embodiment of the present disclosure provides for a system for predicting lower limb movement. The apparatus includes a plurality of sensors, a computing device, at least one movement apparatus, and a controller. The plurality of sensors is located adjacent to indirect muscle groups on a user. The computing device is configured to receive sensor data from the plurality of sensors and determine whether the sensor data comprises a movement pattern. The movement pattern is associated with one of a plurality of movement intentions. The at least one movement apparatus is configured to move a lower limb. The controller causes the at least one movement apparatus to move according to one of the movement intentions, based on determining that the sensor data includes a movement pattern.

Another embodiment of the present disclosure includes an apparatus for activating muscles during cycling. The apparatus includes at least two accelerometers, at least one stimulator, a computing device, and a controller. The at least one stimulator includes a pair of electrodes positioned adjacent to human tissue of the lower limb and determines whether the sensor data includes phasic activity. The controller causes the at least one stimulator to apply a pattern of action potentials to stimulate a cyclic movement of the lower limb.

Additional examples of the above embodiments are described herein.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The accompanying drawings exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1A shows an exemplary modular system according to an embodiment of the present disclosure.

FIG. 1B shows an exemplary modular system for assisting with cyclic activity according to an embodiment of the present disclosure.

FIG. 1C shows an exemplary modular system for clonus prevention according to an embodiment of the present disclosure.

FIG. 2 shows an exemplary wearable module for providing electrical stimulation according to an embodiment of the present disclosure.

FIG. 3A shows an exemplary sensor placement for a wearable module according to an embodiment of the present disclosure.

FIG. 3B shows an exemplary sensor placement for a wearable module according to another embodiment of the present disclosure.

FIG. 4 shows an exemplary movement chart for predictive gait assistance according to an embodiment of the present disclosure.

FIG. 5 shows an exemplary methodology for clonus prevention according to an embodiment of the present disclosure.

FIG. 6A shows exemplary electromyography (EMG) data of an ipsilateral external oblique according to an embodiment of the present disclosure.

FIG. 6B shows exemplary electromyography (EMG) data of a contralateral external oblique according to an embodiment of the present disclosure.

FIG. 6C shows exemplary electromyography (EMG) data of an ipsilateral rectus abdominal according to an embodiment of the present disclosure.

FIG. 6D shows exemplary electromyography (EMG) data of a contralateral rectus abdominal according to an embodiment of the present disclosure.

FIG. 6E shows exemplary electromyography (EMG) data of an ipsilateral erector spinae according to an embodiment of the present disclosure.

FIG. 6F shows exemplary electromyography (EMG) data of a contralateral erector spinae according to an embodiment of the present disclosure.

FIG. 7 shows exemplary inertial movement data for a wheelchair according to an embodiment of the present disclosure.

FIG. 8A shows exemplary inertial movement data for a subject while clonus prevention is not activated.

FIG. 8B shows exemplary inertial movement data for a subject while clonus prevention is not activated.

FIG. 8C shows exemplary inertial movement data for a subject while clonus prevention is not activated.

FIG. 9 is a schematic block diagram illustrating an exemplary system in accordance with an implementation of the present disclosure.

### DETAILED DESCRIPTION

The present invention is described with reference to the attached figures, where like reference numerals are used throughout the figures to designate similar or equivalent elements. The figures are not drawn to scale, and are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details, or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

The present disclosure provides a modular system to assist with lower limb movement. The modular system includes a plurality of wearable modules and a controller. Each wearable module includes a stimulator and a sensor. The controller is configured to detect a desired activity based on data from the sensors on the wearable modules. The controller selects particular wearable modules based on the desired activity and causes the stimulators on those wearable modules to provide electrical stimulation to assist with the desired activity.

The electrical stimulation provided by the wearable modules uses the electrophysiology of a user's muscles and neurons to enable artificial electrical impulses from the stimulators to activate the paralyzed tissues. Therefore, the disclosed system provides directed assistance to particular muscle groups depending on a user's needs. Such a system eliminates the bulkiness of conventional orthosis and also allows a user to develop and use his natural muscles while moving, instead of relying more heavily on artificial assistance. Activating neuromuscular tissue can reduce comorbidities, improve paralyzed tissue health, and even promote regeneration. Additionally, because the disclosed system targets only selected muscle groups based on the desired activity, the disclosed system can provide assistance with gait, cycling, and spasticity suppression with the same unitary system. Additional, non-limiting characteristics and benefits of the disclosed modular system are discussed further herein.

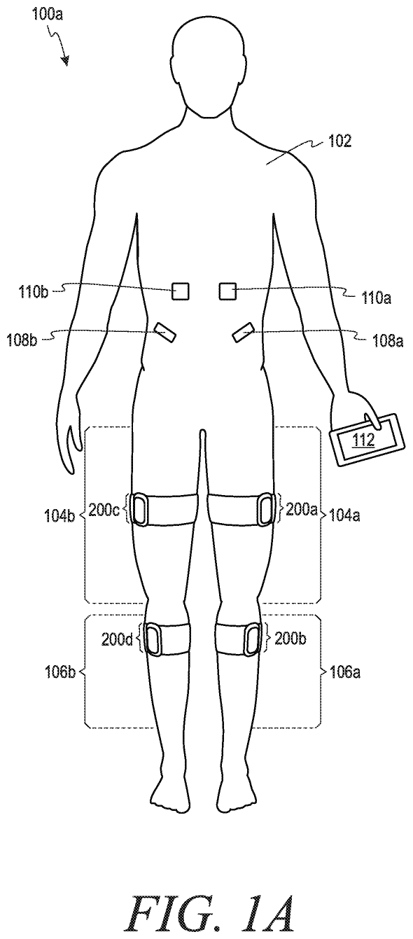

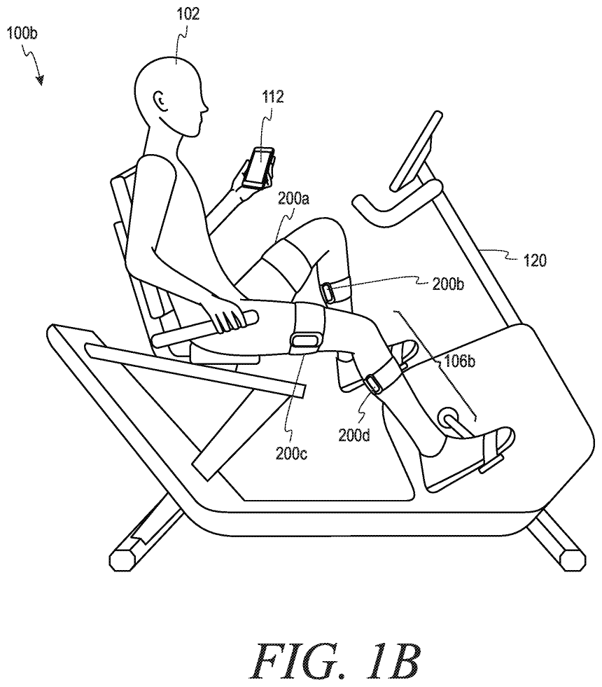

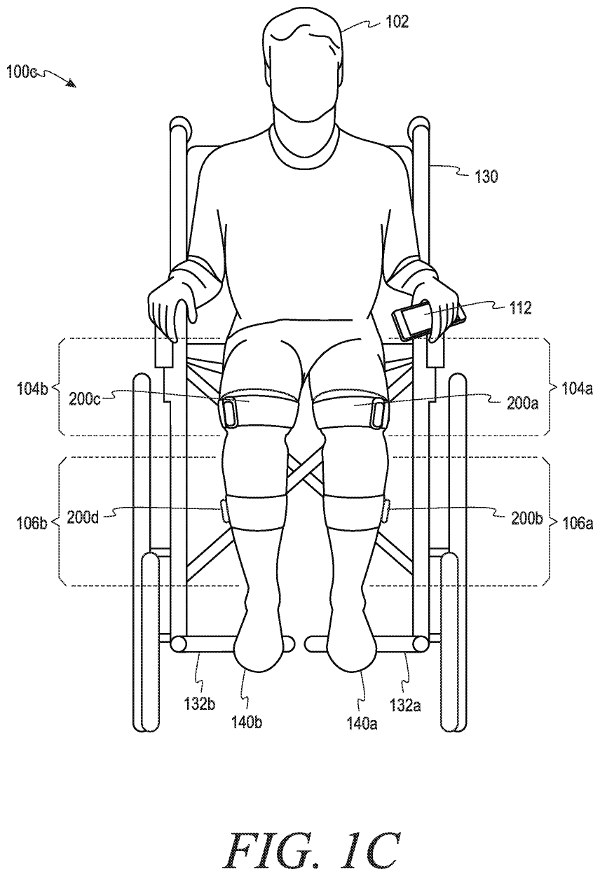

FIG. 1A shows an exemplary modular system 100a according to an embodiment of the present disclosure. The system 100a includes a user 102 with upper leg portions 104a, 104b and lower leg portions 106a, 106b; oblique muscle sensors 108a, 108b; abdominal muscle sensors 110a, 110b; an external device 112; and a plurality of wearable modules 200a-d.

The system 100a includes a plurality of sensors 108a, 108b, 110a, and 110b located on a body of the user 102. The sensors 108a, 108b, 110a, and 110b may be transcutaneous or subcutaneous and may be placed on human tissue of the user 102 along particular muscles. The particular muscles may be chosen based on which muscles move when a user 102 uses his lower limbs. The sensors 108a, 108b, 110a, and 110b are configured to sense movement data of a user 102 and can be communicatively coupled to an external device 112. For example, the sensors 108a, 108b can be located on a user's 102 external oblique muscles, and the sensors 110a, 110b can be located on a rectus abdominal muscles of the user 102. Although certain groups of sensors are shown, additional sensors may be located anywhere on the user 102. For example, the sensors may be located on the erector spinae muscles, additional trunk muscles, and the user's upper leg portions 104a, 104b. The sensors 108a, 108b, 110a, and 110b may be accelerometers, EMG sensors, or any other sensor that can detect activation of the trunk muscles of the user 102.

The system 100a also includes a plurality of wearable modules 200a-d, which can be worn on the user 102. For example, a user 102 can have wearable modules 200a and 200c on upper leg portions 104a, 104b respectively, and wearable modules 200b and 200d on lower leg portions 106a, 106b. Although four wearable modules 200a-d are shown, it is contemplated that any number of wearable modules may be worn on legs of the user 102. Additionally, it is contemplated that each wearable module is capable of selectively activating one or more anatomical sites, according to the placement of the electrodes in the wearable modules.

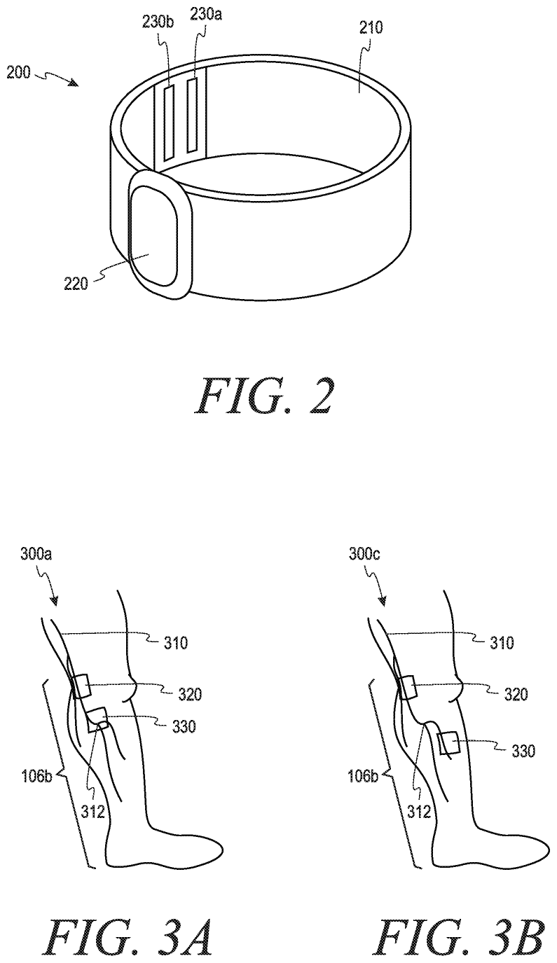

Referring to FIG. 2, an exemplary wearable module 200 (such as wearable modules 200a-d of FIG. 1A) may include a band 210, an electronics enclosure 220, and a pair of electrodes 230a, 230b. The band 210 may secure the module 200 to the user 102 and may be made of a soft, moisture-wicking material for the comfort of the user 102. The electronics enclosure 220 encloses an inertial sensor, a battery, a microcontroller, a printed circuit board (PCB), stimulator circuitry, and a wireless communication module. The inertial sensor is any sensor to sense movement of the wearable module, including an accelerometer, a gyroscope, a magnetometer, a goniometer, an EMG sensor, or any other sensor as known in the art. The controller provides processing of the inertial sensor data and controls the stimulation circuitry on the processing of the inertial sensor data. The stimulation circuitry can control pair of electrodes 230a, 230b based on instructions from the controller. The pair of electrodes 230a, 230b is positioned on an interior surface of the module 200, so as to directly interface with the skin of the user 102.

The pair of electrodes 230a, 230b may be sticky electrodes, sponges, metallic electrodes, or any other electrode type, as known in the art. In some examples, module 200 further includes a cover for the electrodes 230a, 230b to maintain the electrode during storage. The electronics in the enclosure 220 wirelessly receive data from an external device (e.g. another module 200 or external device 112 of FIG. 1). The electrodes 230a, 230b may cause a plurality of action potentials at the neuromuscular tissue underlying the skin of the user 102. The electrodes 230a, 230b provide a selected action potential (or a selected pattern of action potentials) based on instructions from an external device (e.g., thereby providing neuromuscular stimulation). Although one pair of electrodes 230a, 230b and one electronics enclosure 220 are shown in module 200, it is contemplated that any number and type of electrode pairs and sensors can be housed in the module 200. In some examples, sensors are housed along the band 210 outside of the enclosure 220.

The wearable modules 200 may be linked by wired or wireless communication, providing a network of kinematic data to guide a controller. The wearable modules 200 can actuate paralyzed limbs independently, or in tandem.

Additionally, based on data from the inertial measurement sensors, the wearable modules 200 measure the orientation of a limb segment (e.g. limb portions 104a, 104b, 106a, and 106b). When multiple wearable modules 200 are selectively placed on multiple limb segments 104a, 104b, 106a, and 106b, comprehensive data related to body orientation can be provided. With this data and a determined activity mode (as determined by the external device 112 or selected by a user), the wearable modules 200 can actuate the limbs 104a, 104b, 106a, and 106b to assist with movement, provide rehabilitation, and restore function to the paralyzed limb. Additionally, the electrical stimulation can supplement recreational fitness activities for non-paralyzed limbs.

Referring back to FIG. 1A, each wearable module 200a-d may be communicatively coupled to an external device 112. For example, the external device 112 can be a smart phone or a mobile device.

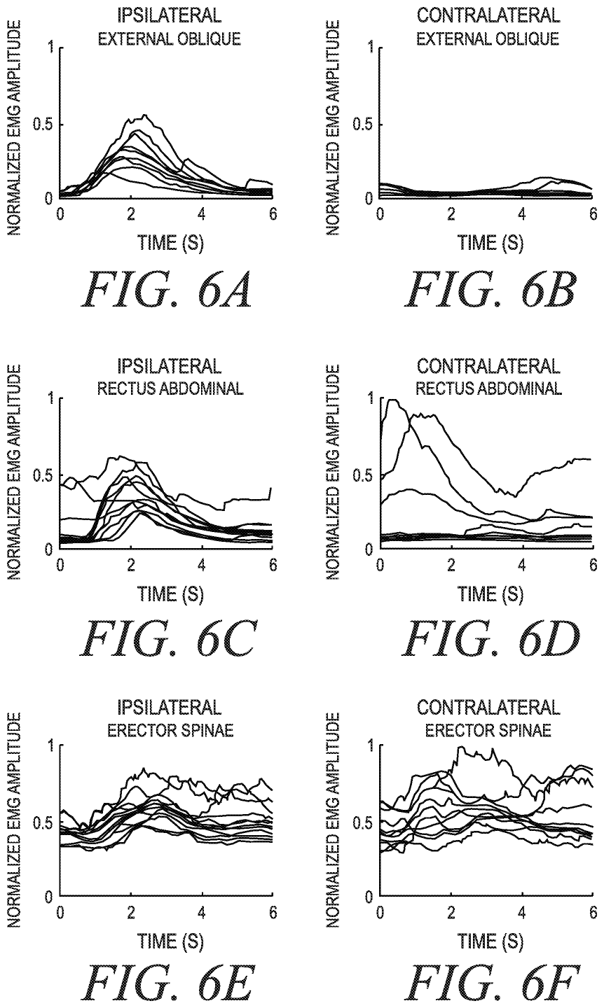

The external device 112 can contain a processor configured to (1) receive data from sensors 108a, 108b, 110a, and 110b and the sensors on each wearable module 200a-d; (2) process the received sensor data; (3) determine a desired activity and a desired activity trait; and (4) instruct the stimulator and electrodes on each wearable module 200a-d to operate, based on the desired activity and the desired activity trait (example processing and operating instructions are discussed further below with respect to FIGS. 5 and 6). For example, the external device 112 may detect that the user 102 intends to take a right step; the external device 112 then actuates modules 200c, 200d to provide electrical stimulation that encourages contraction and relaxation of the appropriate muscles for walking. In another example, the external device 112 may detect that the user 102 is cycling or participating in another activity with phasic movement; the external device 112 then actuates modules 200a-d to provide electrical stimulation that encourages continuous phasic activity of the appropriate muscles to continue the activity. Additional examples are discussed further below, with respect to FIGS. 1B, 1C, 5, and 6. In some examples, steps (1), (2), and (3) performed by the processor of the external device 112 are performed by the processors on the individual wearable modules 200a-d.

Thereby, the wearable modules 200a-d can provide a modular functional electronic system (FES), which measures movement data (sensors on modules 200a-d) or movement intention data (sensors 108a, 108b, 110a, and 110b). The modular nature of the disclosed system 100a allows individual modules 200a-d to be activated according to which limb portion 104a, 104b, 106a, and 106b requires movement assistance. For example, if a user wishes to take a right step, only limb portions 104a, 106a need to move, so the external device 112 instructs wearable modules 200a, 200b to provide appropriate electrical stimulation.

In some embodiments of the system 100a, the wearable modules 200a-d can be prosthetics, mechanically-actuated orthoses, or other movement apparatus, as known in the art, to enable movement of the lower limbs of the user 102.

In some examples of the system 100a, the external device 112 can include an interface. The interface can receive a selection of a desired activity (for example, a selection by user 102) and can actuate wearable modules 200a-d, according to the selected activity.

In some examples of system 100a, the external device 112 includes a supervisory controller and a low-level controller. The supervisory controller determines a desired activity based on sensor data (e.g., walking, sit-to-stand, stand-to-sit, cycling, clonus preventions, etc.). A low-level controller provides functional electrical stimulation (FES) based on the desired trait and the sensor data. For example, the low-level controller is the controller in any of the wearable modules 200a-d.

In some examples of system 100a, the external device 112 includes a supervisory controller and communicate settings to the wearable modules 200a-d that employ a low-level controller to autonomously control stimulation behavior.

In some examples, the external device 112 determines a stimulation amplitude for the electrodes in wearable modules 200a-d. The stimulation amplitude may be based on sensor data, the desired activity, user preference, and any trait of the desired activity.

FIG. 1B shows an exemplary modular system 100b for assisting with cyclic activity according to an embodiment of the present disclosure. System 100b can include similar components and labeling as system 100a of FIG. 1A. System 100b also includes a cycling-powered mechanism 120. In system 100b, modules 200a, 200c each contain at least one accelerometer.

The user 102 of FIG. 1B is operating the mechanism 120. The user 102 has an inertial sensor containing at least one accelerometer on the wearable modules 200a, 200c of each upper leg portion 104a, 104b. These inertial sensors are communicatively coupled to the external device 112. The external device 112 is configured to detect phasic activity of the user 102 based on the accelerometer data provided by wearable modules 200a-d. When phasic activity is detected, the external device 112 can coordinate with the wearable modules 200a-d to provide a pattern of action potentials to continue and stimulate a cyclic movement of the lower limbs. In some examples of the present disclosure, wearable modules 200a and 200c can each contain two accelerometers. The two accelerometer values may then be orientated and processed to convey angular orientation of the module.

System 100b thereby provides a modular system, worn by a user 102, to assist with cycling activity. Although a stationary bike is shown in FIG. 1B, any cycling-powered mechanism can be used by the user 102, including bicycles, unicycles, pedal-boats, and other pedaled-vehicles. System 100b does not require wires of a conventional cycling assistance device. Conventional devices are typically plugged directly into a mechanism 120, attached to pedals instead of directly to a user 102. Therefore, by the disclosed modular system, the user 102 can use any cycling device, even if it is not equipped for a person with neurological impairment.

FIG. 1C shows an exemplary modular system 100c for clonus prevention according to an embodiment of the present disclosure. System 100c may include similar components and labeling as system 100a. System 100c also includes a wheelchair 130, footrests 132a, 132b, and a user's feet 140a, 140b.

Clonus can be triggered by involuntary reflexes or by bumpiness of terrain as a wheelchair user moves. When clonus occurs, the feet 140a, 140b of a user 102 typically seize up, oscillate at the ankle, and fall off footrests 132a, 132b, respectively. When the feet 140a, 140b fall off of the footrests 132a, 132b, the feet 140a, 140b can be dragged under the wheelchair 102 against the ground, causing injury and/or discomfort to the user.

System 100c provides means for preventing clonus from occurring and ending clonus quickly if it has occurred. Wearable modules 200a-d are positioned adjacent to a muscle tissue of a user 102 and each have one or more sensors to collect data on limb movement. In some examples, sensors detect clonus based on identifying the relative motion of the clonus-affected limb to a stationary point of the body 102 or wheelchair 130. The wearable modules 200b, 200d process the data to determine whether it comprises a frequency characteristic of the clonus reflex. Prominent oscillations within 3-8 Hz indicate that clonus is occurring. When the data comprises clonus activity, the controller in wearable modules 200b, 200d actuates the stimulators on wearable modules 200b, 200d to provide electrical stimulation causing ankle dorsiflexion on the limb portion where clonus was detected. The electrical stimulation may continue until the sensors on 200b, 200d detect ankle dorsiflexion and no subsequent clonus.

In some examples, wearable modules 200a-d also include accelerometers. Accelerometers detect the bumpiness of the wheelchair 130 movement. In some examples, the accelerometers are aligned with the axis of clonus perturbation for the user 102. The wearable modules 200b, 200d receive and process the accelerometer data. The wearable modules 200b, 200d determine a computed inertial measurement based on the accelerometer data. The wearable modules 200b, 200d determine whether the computed inertial measurement is above a first threshold (identifying an amount of movement that triggers clonus) or above a second threshold (identifying an amount of movement that indicates clonus is occurring), and can also determine whether the frequency content is characteristic of wheelchair motion, or clonus. Accordingly, the wearable modules 200b, 200d actuate the enclosed stimulators to provide electrical stimulation causing neural modulation on the limb portion where clonus was detected or was predicted to occur.

Referring to FIGS. 3A-3B, exemplary electrode positions 300a-b are shown on a lower leg portion 106b. These positions 300a-b are selected based on how a user responds to the stimulation caused by external device 112. The lower leg portions 106b of FIGS. 3A-3B includes a tibial nerve 310 with branching 312; a first electrode 320; and a second electrode 330. Electrical stimulation typically uses an artificial charge-balanced electrical impulse to activate human electrophysiology for a response. The two electrodes 320-330 complete the artificial circuit and impose an electric field across underlying anatomy. Adjustments in the placement of electrodes 320, 330 (as shown by FIGS. 3A-3B) alter the response efficacy. Additionally, adjusting stimulation characteristics can alter the response efficacy.

Position 300a shows a placement of first electrode 320 behind a user's knee along the tibial nerve 310 and a placement of second electrode 330 at the tibial nerve branching 312. Position 300a can provide a lower amount of electrical stimulation to a user's muscles; this is particularly beneficial for a user who is quickly responds to electrical stimulation with ankle dorsiflexion.

Position 300b shows a placement of first electrode 320 behind a user's knee along the tibial nerve 310 and farther down on the lower leg portion 106b. Position 300b can provide a high amount of electrical stimulation to a user's muscles; this is particularly beneficial for a user who responds slowly to electrical stimulation or has particularly violent spasms.

In some additional examples, the electrodes 320, 330 are in a wearable module (not pictured) and are flush against the human tissue of a user when the wearable module is worn. In other examples, electrodes 320, 330 are transcutaneous electrodes located adjacent to at least one of the common peroneal nerve and the tibialis anterior nerve.

Therefore, systems 300a-b demonstrate how wearable modules 200 can be worn in a variety of locations on lower leg portions 106a, 106b. Different locations change the positioning of electrodes 320 and 330 along nerve paths and against muscle groups to correspondingly increase or decrease the effect that electrical stimulation has on a user 102. Systems 100c and 300a-300b provide mechanisms to (1) predict when clonus will occur based on bumpiness of terrain levels, (2) provide a neuromodulation stimulation that prevents clonus onset and (3) stop clonus once it has occurred.

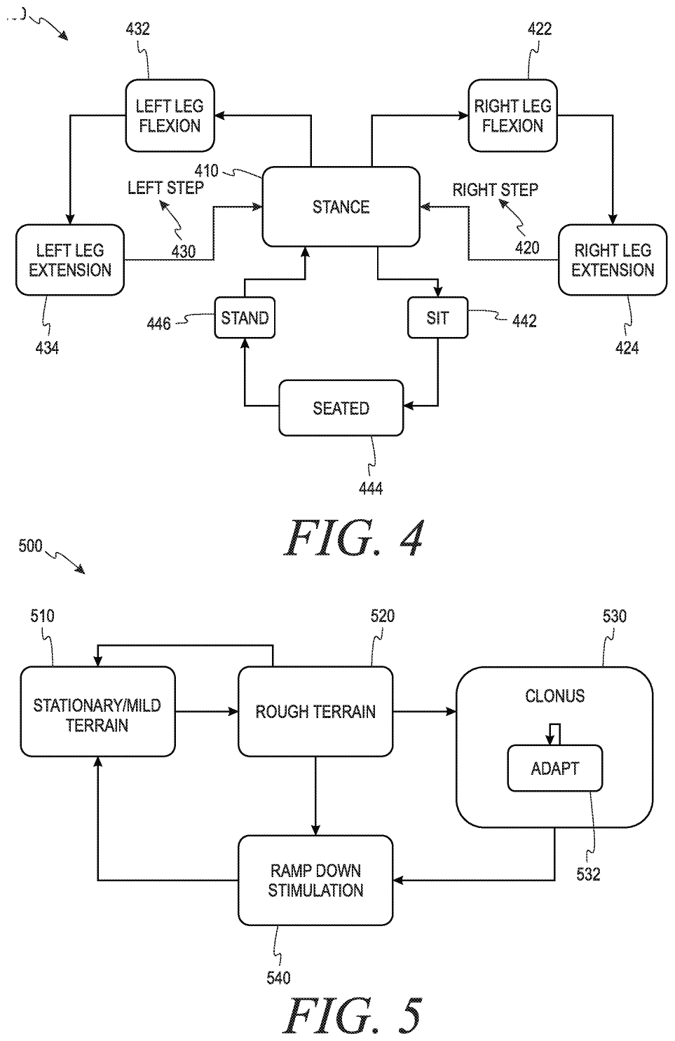

FIG. 4 shows an exemplary movement methodology 400 for predictive gait assistance while using the modular lower limb system 100a of FIG. 1A. Methodology 400 begins when a user is in a stance state 410. Sensors (such as sensors 108a, 108b, 110a, 110b or any other sensors as provided for with respect to FIG. 1A) on a trunk portion of a user 102 can indicate movement intention. In some examples, these sensors can be EMG sensors. Movement intention is determined by an external device 112 that receives the EMG data and determines whether the EMG data provides a pattern consistent with a particular movement (e.g. a right step 420, a left step 430, or a stand-to-sit transition 440) in one embodiment. This determination can happen based on machine learning on how a particular user 102 moves, or based on comparing current EMG sensor data to a database of movement intention EMG data.

For example, if the external device 112 determines that the EMG data indicates a user 102 intention to take a right step 420 (e.g., because sensors 108b and/or 110b detect a movement pattern consistent with intending to move a right leg), the external device 112 activates electrical stimulation units on wearable modules 200c, 200d. The stimulators cause action potentials on the user's upper leg portion 104b and lower leg portion 106b, respectively. This action potential causes a right leg flexion 422, a subsequent right leg extension 424, before bringing the user back to a stance state 410. Similarly, determining that the EMG data indicates an intention to take a left step 430, the external device 112 can activate electrical stimulation units on wearable modules 200a, 200b; the stimulators cause action potentials on the user's upper leg portion 104a and lower leg portion 106a, respectively, and this action potential causes a left leg flexion 432, a subsequent left leg extension 434, before bringing the user back to a stance state 410.

The methodology 400 also detects, based on sensor data, when a user 102 wants to perform a sit transition 442 to be seated 444, and when a user 102 wants to perform a stand transition 446 to return to a stance state 410. From a seated position 444, the external device 112 activates extensor stimulation based on orientation data received from sensors on wearable modules 200a, 200c. For example, the external device 112 activates the stimulation when either the thigh angle or angular velocity of the thigh exceeds a threshold, indicating the user has initiated a sit-to-stance maneuver 446. The extension stimulation can be applied to the quadriceps and/or gluteus maximus, via wearable modules 200a, 200c. The stimulation parameters can be predefined, or varied using a transform dependent on input signals from the sensors 108-110 and inertial sensors on wearable modules 200a-d. The stimulation may be controlled so that the thigh angle follows a predefined angle trajectory or angular velocity trajectory. After the angle increases past a predefined threshold, the external device 112 transitions to a stance state 410. In the stance state 410, the external device 112 ramps down the extensor stimulation of wearable modules 200a and 200c or maintains extension for stance support.

When transitioning from stand-to-sit 442, the external device 112 determines if the sensor data on wearable modules 200a, 200c indicate that the user 102 is leaning back (or, e.g., detecting that the thigh orientation has reclined). When the user reaches the sit state 442, the extensor stimulation at wearable modules 200a, 200c is set to a predefined parameter setting or a closed loop feedback, such that the thigh is encouraged to follow a predefined kinematic trajectory. Once the thigh angle is below a final threshold, the controller transitions to the seated state 444, and the extensor stimulation at wearable modules 200a and 200c may be transitioned off.

In some examples, methodology 400 uses an actuated mechanical orthosis instead of, or in addition to, wearable modules 200. In some examples, methodology 400 can rely on sensor data from sensors located anywhere on indirect muscle groups of a user 102. Indirect muscle groups include, for example, rostral muscle groups unaffected by neurological injury and oblique muscle groups (or any other muscles which do not carry the body while walking, but which provide indirect movement support). In some examples, the sensors use transcutaneous electrodes located adjacent to muscle tissue of the indirect muscle groups.

System 100a of FIG. 1A, when combined with methodology 400, predicts when a user wants to take a step without requiring movement of a lower limb. Conventional designs either require user input or leg movement to make any of the transitions of methodology 400 (conventional designs typically place sensors directly on the human leg or the mechanical orthosis). By contrast to conventional designs, the disclosed system 100a and methodology 400 detects intention from indirect muscle groups without requiring any actual movement of the leg portions 104a, 104b, 106a, and 106b. This provides a far greater amount of assistance, especially for users who are unable to initiate movement.

FIG. 5 shows an exemplary methodology 500 for clonus prevention while using the modular lower limb system 100c of FIG. 1C. A user 102 may be in any of the states of FIG. 5, and the user's particular state is determined by the external device 112 or by the wearable modules 200a-d based on accelerometer data (for example, from accelerometers on wearable modules 200a-d) or other sensor data. For example, sensor data indicating a stretch reflex identifies that the user 102 is in clonus state 530.

The accelerometer data is analyzed by the wearable modules 200a-d or an external device (e.g. device 112) which processes the signal to produce a computed inertial measurement. The accelerometer signal is generally interpreted with discrete logic, or processed by filters and interpreted by a threshold-based protocol to identify when clonus is occurring. For example, a bandpass filter may be applied to the accelerometer signal from 3-8 Hertz (hz); this selects a frequency band that is typically characteristic of clonus. The signal is then rectified and low-pass filtered to obtain a computed inertial measurement, which characterizes clonus activity. In some examples, the accelerometer data is separated into a first and second frequency range. For example, data in the 3-8 Hz range makes up a first frequency range, which corresponds to limb movement due to clonus. The remaining data makes up a second frequency range, which corresponds to limb movement due to external excitation (such as wheelchair motion over uneven terrain).

Another processing method provides for isolating the clonus frequency band data from the accelerometer by using a Fast Fourier Transform (FFT). This method is highly selective for specifying clonus after analyzing at least two clonus occurrences. Alternative pattern recognition algorithms (e.g. principal component analysis) may be employed by Method 500 for classifying sensor data. Method 500 may be provided for in either an open loop configuration or in a discrete state machine controller when external device 112 determines whether to apply a ramp down stimulation 540.

In some examples of methodology 500, two thresholds may be provided for to determine whether the user 102 is in a non-clonus state 510, a clonus-inducing state 520, or a clonus state 530. For example, if the user 102 is experiencing rough terrain 520, the external device 112 determines based on the processing of the sensor data that the computed inertial measurement is above a first threshold (signifying clonus-inducing movement of state 520), but below a second threshold (signifying a clonus state 530). When the computed inertial measurement is between the two thresholds, the external device may either (1) perform no stimulation—where the user 102 waits to experience mild terrain or transition to a clonus state 530—or (2) perform a rough terrain stimulation protocol and then enact the ramp down stimulation 540. The rough terrain stimulation protocol provides electrical stimulation to the designated wearable module to prevent clonus from occurring in response to the rough terrain. When the clonus state 530 is detected (clonus state 530 is automatically detected when the computed inertial measurement rises above the second threshold), the controller automatically provides electrical stimulation designed to end clonus. The ramp down stimulation 540 is automatically enacted after the clonus state 530 is no longer detected.

The electrical stimulation caused by methodology 500 in the rough terrain state 520, the clonus state 530, and the ramp down stimulation 540 is applied via a pair of electrodes for a selected period of time (for example in electrodes 230a, 230b of module 200). The current level of the electrodes and the time period of the applying can be based on the computed inertial measurement. An external device 112 or a processor in wearable modules 200b, 200d determines whether the computed inertial measurement has fallen below the first threshold before continuing to apply stimulation via the electrodes 230a, 230b. In some examples, a user 102 sets the first and second threshold to be particular to the user's data. For example, a user 102 provides a reference for what is clonus-induced motion versus terrain-induced motion.

In some examples, intermittent stimulation is provided to the lower leg portions 106a, 106b.

If the external device 112 takes no action when in state 520, the external device 112 continues to monitor whether the user 102 switches into another state. In some examples, the user may continue to experience rough terrain 530 without triggering clonus until the user begins to experience a mild terrain or stationary position 520. In other examples, the rough terrain eventually triggers clonus 530. In some examples of methodology 500, a user may experience clonus 530 without a perceptible trigger from rough terrain 520.

Performing a ramp down stimulation 540 comprises reducing the electrically stimulating amplitude to none in the appropriate lower leg portions 106a, 106b via wearable modules 200b, 200d. The electrical stimulation during the rough terrain 520 and clonus states 530 stimulates the lower leg portions 106a, 106b into a withdrawal reflex, or ankle dorsiflexion. In some examples, the stimulation amplitude is incremented slowly until the computed inertial measurement drops significantly below the second threshold within an allotted time (i.e. if a clonus state 530 persists beyond four seconds of anti-clonus stimulation, the controller of the appropriate wearable module 200b, 200d increments the amplitude in adapt sub-state 532). Typical stimulation parameters are biphasic waveforms, (e.g. 15-60 Hz, 10-160 mA) to activate neurons and musculature. High frequency carrier frequencies are also used to induce neuromodulation.

In some examples of methodology 500, transitions between states occur based on user input.

In some examples of methodology 500, intermittent stimulation is provided to lower limb portions 106a and 106b. Intermittent stimulation generally improves blood flow and prevents pressure sores common to users who are confined to wheelchairs.

Overall, the use of thresholds and separating the accelerometer data into separate frequency ranges allows method 500 to detect when the accelerometer signal data includes just movement of the wheelchair 130 across bumpy terrain, or whether the data includes disturbance caused by clonus.

Altogether, FIGS. 1-5 show exemplary systems, methods, and electrode placements that can be used independently or in any combination to assist users with neurological impairments affecting lower limb movement. In particular, an exemplary system that makes use of all the functions above is much more desirable to conventional devices, because the disclosed system can seamlessly switch between activities based on sensor data, and without any input from the user.

### Experimental Data

FIGS. 6A-6F show exemplary electromyography (EMG) data for predicting step intention of a user, according to embodiments of the present disclosure. Each graph shows a plurality of normalized EMG measurements taken over time as a user attempts to take a step. Each graph shows the pattern of EMG measurements at that muscle location over time. For example, if an external device (e.g. device 112) were to continuously receive EMG data from the regions of FIGS. 6A-6F, the external device 112 predicts when the user intends to take a step, based on whether the present EMG data matches any of the EMG patterns in a database.

FIGS. 6A-6B show EMG measurements of muscle movement for external oblique muscles. Each separate line shows a different step. FIGS. 6A-6B demonstrate that ipsilateral oblique measurements provide a clear pattern indicating when a user intends to take a step, while contralateral muscles provide a much more muted response. Additionally, the obliques activity level may convey information on step characteristics such as: step size, step speed, and step duration.

FIGS. 6C-6D respectively show ipsilateral and contralateral EMG measurements for a plurality of steps, as measured by sensors on the rectus abdominal muscles. FIGS. 6E-6F respectively show ipsilateral and contralateral EMG measurements for a plurality of steps as measured by sensors on the erector spinae muscles of a user. Although FIGS. 6C-6F show a less clear pattern than the pattern demonstrated in FIG. 6A, the present disclosure contemplates that each user can have a particular EMG pattern that can be learned by an external device over time (e.g., via machine learning).

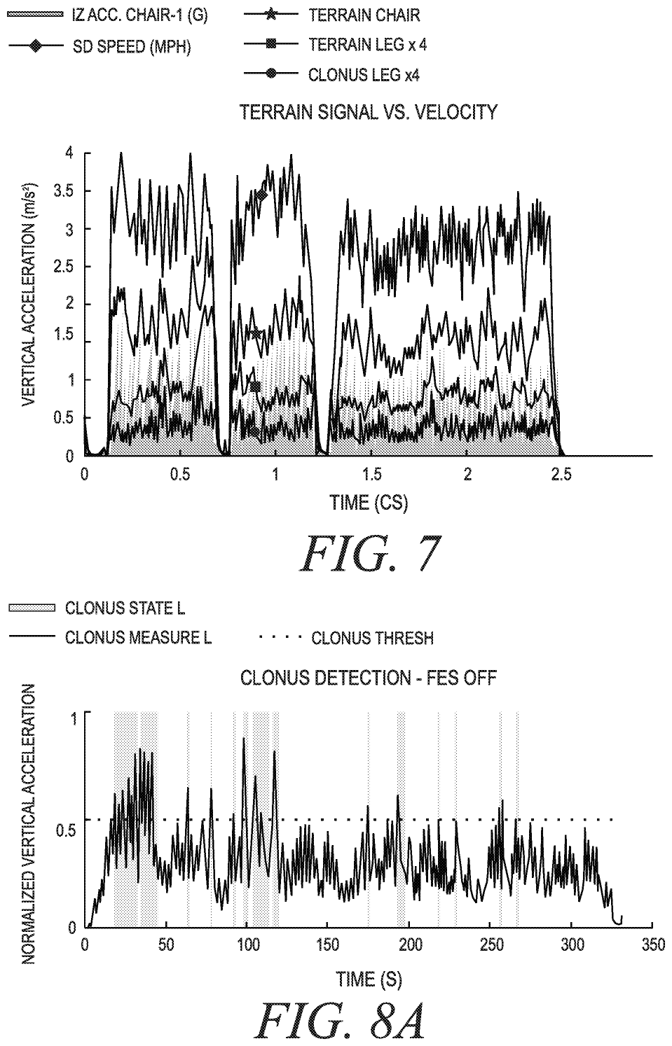

FIG. 7 shows exemplary inertial movement data for a wheelchair according to an embodiment of the present disclosure. The inertial movement data was captured via real-time velocity measurements and was synchronously parsed with inertial sensors mounted on the wheelchair. The terrain signal data from the chair-mounted inertial sensors had minimal clonus interference and was found to correlate greatest with the measured speed, resulting in a Pearson correlation coefficient of 0.85. The terrain signal and clonus signal from an exemplary leg (right) had a correlation coefficient of 0.5 and 0.43 with the measured speed respectively. This indicates that mounted inertial sensors and specific signal processing can be used to estimate speed over terrain.

P-values of zero indicated there is positive correlation between the filtered vertical acceleration from both legs and the chair. Therefore, FIG. 7 shows that the vertical accelerometer signal feasibly indicates movement over a rough terrain. The terrain leg lines in FIG. 7 show that the legs absorb vibrations from the ground. While the terrain signal from a chair mounted IMU provides the greatest correlation to velocity, any of IMUs correlate sufficiently to detect binary motion (moving or not moving).

FIG. 7 also shows that terrain measurement is prone to interference from clonus. For example, clonus of one leg can cause mild interference with terrain measurement of the contralateral leg and chair (as these units are physically interfaced through the chair frame and vertically bound by the ground). A notch filter can be used to remove the interference caused by the clonus signal. Therefore, FIG. 7 shows that identifying frequency ranges for clonus and rough terrain detection, as discussed with respect to FIG. 5 above, increases the accuracy for determining when to enact a ramp down stimulation pattern.

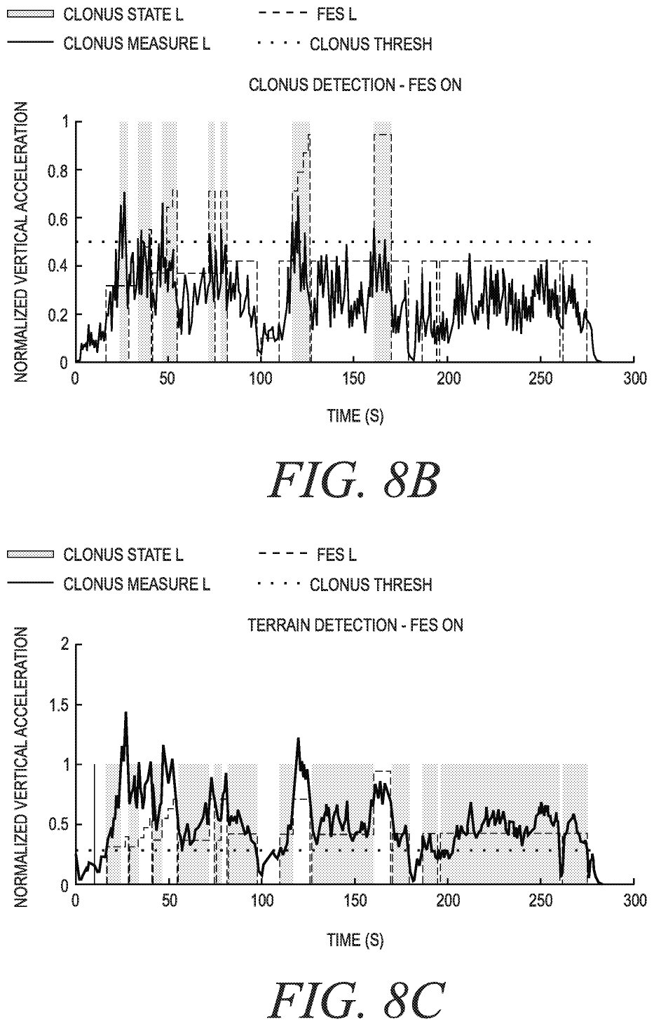

FIG. 8A shows accelerometer data over a period of time for a leg, where clonus is identified whenever the signal goes above the dotted line at 0.5 (also shown in the shaded region). FIG. 8A shows user data for a user without activation of stimulation by the disclosed wearable modules. FIG. 8A demonstrated that, when unchecked, clonus will continue to trigger repeatedly over a period of several minutes.

FIG. 8B shows accelerometer data over a period of time for a leg which includes a wearable module 200. The dotted line demonstrates the electrical stimulation amplitude which was applied in response to clonus detection (when the signal rose above 0.5), according to an embodiment of the present disclosure. The shaded region shows when the clonus state was detected. FIG. 8B further demonstrated that, when using a wearable module 200, the user experienced less time in a clonus state (as compared against the clonus-state data of FIG. 8A).

FIG. 8C shows accelerometer data over a period of time for a leg that includes a wearable module 200, which activates FES when rough terrain is detected (before a clonus-state is caused by the rough terrain). This embodiment serves to prevent clonus from ever occurring in response to rough terrain. The shaded region demonstrates when preventative electrical stimulation is engaged and the dotted line shows the normalized electrical stimulation amplitude which was applied. FIG. 8C shows that the disclosed wearable modules 200 can react to rough terrain as well as clonus detection.

### Computer Hardware

FIG. 9 is a schematic block diagram illustrating an exemplary server system 900, in accordance with an implementation of the present disclosure. In this example, the server system 900 includes at least one microprocessor or processor 904; a BMC 903; one or more cooling modules 960; a main memory (MEM) 911. At least one power supply unit (PSU) 902 that receives an AC power from an AC power supply 901, and provides power to various components of the server system 900, such as the processor 904, north bridge (NB) logic 906, PCIe slots 960, south bridge (SB) logic 908, storage device 909, ISA slots 950, PCI slots 970, and BMC 903.

After being powered on, the server system 900 is configured to load software application from memory, a computer storage device, or an external storage device to perform various operations. The storage device 909 is structured into logical blocks that are available to an operating system and applications of the server system 900. The storage device 909 is configured to retain server data even when the server system 900 is powered off.

In FIG. 9, the memory 911 is coupled to the processor 904 via the NB logic 906. The memory 911 may include, but is not limited to, dynamic random access memory (DRAM), double data rate DRAM (DDR DRAM), static RAM (SRAM), or other types of suitable memory. The memory 911 can be configured to store firmware data of the server system 900. In some configurations, firmware data can be stored on the storage device 909.

In some implementations, the server system 900 can further comprise a flash storage device. The flash storage device can be a flash drive, a random access memory (RAM), a non-volatile random-access memory (NVRAM), or an electrically erasable programmable read-only memory (EEPROM). The flash storage device can be configured to store system configurations such as firmware data.

The processor 904 can be a central processing unit (CPU) configured to execute program instructions for specific functions. For example, during a booting process, the processor 904 can access firmware data stored in the BMC 903 or the flash storage device, and execute the BIOS 905 to initialize the server system 900. After the booting process, the processor 904 can execute an operating system in order to perform and manage specific tasks for the server system 900.

In some configurations, the processor 904 can be multi-core processors, each of which is coupled together through a CPU bus connected to the NB logic 906. In some configurations, the NB logic 906 can be integrated into the processor 904. The NB logic 906 can also be connected to a plurality of peripheral component interconnect express (PCIe) slots 960 and an SB logic 908 (optional). The plurality of PCIe slots 960 can be used for connections and buses such as PCI Express x1, USB 2.0, SMBus, SIM card, future extension for another PCIe lane, 1.5 V and 3.3 V power, and wires to diagnostics LEDs on the server system 900's chassis.

In system 900, the NB logic 906 and the SB logic 908 are connected by a peripheral component interconnect (PCI) Bus 907. The PCI Bus 907 can support functions on the processor 904 but in a standardized format that is independent of any of the processor 904's native buses. The PCI Bus 907 can be further connected to a plurality of PCI slots 970 (e.g., a PCI slot 971). Devices connect to the PCI Bus 907 may appear to a bus controller (not shown) to be connected directly to a CPU bus, assigned addresses in the processor 904's address space, and synchronized to a single bus clock. PCI cards that can be used in the plurality of PCI slots 970 include, but are not limited to, network interface cards (NICs), sound cards, modems, TV tuner cards, disk controllers, video cards, small computer system interface (SCSI) adapters, and personal computer memory card international association (PCMCIA) cards.

The SB logic 908 can couple the PCI Bus 907 to a plurality of expansion cards or ISA slots 950 (e.g., an ISA slot 951) via an expansion bus. The expansion bus can be a bus used for communications between the SB logic 908 and peripheral devices, and may include, but is not limited to, an industry standard architecture (ISA) bus, PC/904 bus, low pin count bus, extended ISA (EISA) bus, universal serial bus (USB), integrated drive electronics (IDE) bus, or any other suitable bus that can be used for data communications for peripheral devices.

In this example, BIOS 905 can be any program instructions or firmware configured to initiate and identify various components of the server system 900. The BIOS is an important system component that is responsible for initializing and testing hardware components of a corresponding server system. The BIOS can provide an abstraction layer for the hardware components, thereby providing a consistent way for applications and operating systems to interact with a peripheral device such as a keyboard, a display, and other input/output devices.

In system 900, the SB logic 908 is further coupled to the BMC 903 that is connected to the PSU 902. In some implementations, the BMC 903 can also be a rack management controller (RMC). The BMC 903 is configured to monitor operation status of components of the server system 900, and control the server system 900 based upon the operation status of the components.

Although only certain components are shown within the exemplary systems 900 in FIG. 9, various types of electronic or computing components that are capable of processing or storing data, or receiving or transmitting signals, can also be included in the exemplary system 900. Further, the electronic or computing components in the exemplary system 900 can be configured to execute various types of application, and/or can use various types of operating systems. These operating systems can include, but are not limited to, Android, Berkeley Software Distribution (BSD), iPhone OS (iOS), Linux, OS X, Unix-like Real-time Operating System (e.g., QNX), Microsoft Windows, Window Phone, and IBM z/OS.

Depending on the desired implementation for the exemplary systems 900, a variety of networking and messaging protocols can be used, including but not limited to TCP/IP, open systems interconnection (OSI), file transfer protocol (FTP), universal plug and play (UpnP), network file system (NFS), common internet file system (CIFS), AppleTalk etc. As would be appreciated by those skilled in the art, FIG. 9 is used for purposes of explanation. Therefore, a network system can be implemented with many variations, as appropriate, yet still provide a configuration of network platform in accordance with various examples of the present disclosure.

In exemplary configurations of FIG. 9, the exemplary system 900 can also include one or more wireless components operable to communicate with one or more electronic devices within a computing range of the particular wireless channel. The wireless channel can be any appropriate channel used to enable devices to communicate wirelessly, such as Bluetooth, cellular, NFC, or Wi-Fi channels. It should be understood that the device can have one or more conventional wired communications connections, as known in the art. Various other elements and/or combinations are possible as well within the scope of various examples.

While various examples of the present invention have been described above, it should be understood that they have been presented by way of example only, and not limitation. Numerous changes to the disclosed examples can be made in accordance with the disclosure herein without departing from the spirit or scope of the invention. Thus, the breadth and scope of the present invention should not be limited by any of the above described examples. Rather, the scope of the invention should be defined in accordance with the following claims and their equivalents.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The terminology used herein is for the purpose of describing particular examples only and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this invention belongs. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

1. A system for assisting lower limb movement, the system comprising:
at least one wearable module including:
at least one stimulator including a pair of electrodes positioned adjacent to human tissue of a lower limb, the at least one stimulator configured to cause a plurality of action potentials at the human tissue; and
at least one inertial sensor configured to receive inertial data of the at least one wearable module; and
a controller communicatively coupled to the at least one stimulator and the at least one inertial sensor, the controller configured to:
determine a desired activity and at least one trait of the desired activity based on the inertial data; and
apply at least one action potential from the plurality of action potentials to the human tissue, via the at least one stimulator, for a selected duration, based on the desired activity, the at least one trait, and the inertial data.

2. The system of claim 1, wherein the desired activity includes clonus suppression, and wherein applying the at least one action potential comprises stimulating a nerve that modulates the clonus reflex pathway.

3. The system of claim 1, wherein the at least one inertial sensor comprises two or more accelerometers.

4. The system of claim 3, wherein the desired activity includes cyclic motion assistance, and wherein applying the at least one action potential comprises applying a synchronous pattern of action potentials to stimulate a cyclic movement of the lower limb.

5. The system of claim 1, wherein the desired activity includes gait assistance, and wherein operating the at least one stimulator comprises stimulating the lower limb to take a step.

6. The system of claim 1, wherein the system further comprises a muscle sensor on a human trunk muscle and wherein the controller is further configured to:
receive movement data from the muscle sensor; and
determine whether the desired activity includes gait assistance based on the received movement data.

7. The system of claim 1, wherein the controller is further configured to determine a stimulation amplitude for the at least one stimulator.

8. The system of claim 1, further comprising an interface communicatively coupled to the controller, wherein the interface is configured to:
receive a selection of the desired activity; and
communicate the selection to the controller.

9. An apparatus for reducing pathological lower limb oscillation, the apparatus comprising:
at least one accelerometer positioned adjacent to a lower limb;
a computing device configured to:
receive sensor data from the at least one accelerometer;
process the sensor data to provide a computed inertial measurement; and
determine when the computed inertial measurement passed a first threshold level;
an electrical stimulation unit configured to stimulate ankle dorsiflexion of the lower limb; and
a controller communicatively coupled to the computing device and configured to activate the electrical stimulation unit based on whether the computed inertial measurement passed the first threshold inertial level.

10. The apparatus of claim 9, wherein the computing device is further configured to classify the sensor data as a stretch reflex.

11. The apparatus of claim 9, wherein stimulating the lower limb in ankle dorsiflexion further comprises applying a current via the electrical stimulation unit to the lower limb.

12. The apparatus of claim 9, the computing device further configured to classify the sensor data as non-clonus when the computed inertial measurement is below the first threshold inertial level.

13. The apparatus of claim 9, the computing device further configured to classify the sensor data as clonus-inducing when the computed inertial measurement is at or above the first threshold inertial level and below a second threshold inertial level.

14-15. (canceled)

16. The apparatus of claim 9, wherein the electrical stimulation unit comprises transcutaneous electrodes located adjacent to at least one of the common peroneal nerve and the tibialis anterior nerve.

17. The apparatus of claim 9, wherein the electrical stimulation unit is located external to the lower limb and adjacent to human tissue on the lower limb near at least one of the common peroneal nerve and the tibialis anterior nerve.

18. The apparatus of claim 9, wherein processing the sensor data further comprises separating the sensor data into a first frequency range and a second frequency range, wherein the first frequency range corresponds to limb movement due to clonus, and wherein the second frequency range corresponds to limb movement due to external excitation.

19. The apparatus of claim 18, wherein the external excitation comprises wheelchair motion over uneven terrain.

20. (canceled)

21. A system for predicting lower limb movement, the apparatus comprising:
a plurality of sensors located adjacent to indirect muscle groups;
a computing device configured to receive sensor data from the plurality of sensors, the computing device further configured to determine whether the sensor data comprises a movement pattern associated with one of a plurality of movement intentions;
at least one movement apparatus configured to move a lower limb; and
a controller configured to cause the at least one movement apparatus to move according to one of the movement intentions when the computing device determines that the sensor data comprises a movement pattern.

22-24. (canceled)

25. The apparatus of claim 21, wherein the indirect muscle groups comprise at least one of external oblique muscles.

26. The apparatus of claim 21, wherein the electrical stimulation unit comprises transcutaneous electrodes located adjacent to muscle tissue of the indirect muscle groups.

27. (canceled)
